(19) United States
(12) Patent Application Publication
(10) Pub. No.: US 2024/0361237 A1
(43) Pub. Date: Oct. 31, 2024

(54) INTERFEROMETRIC ELEMENT, DEVICE FOR DETECTING A COMPOUND COMPRISING AN INTERFEROMETRIC ELEMENT AND METHOD FOR DETECTING A COMPOUND

(71) Applicant: COMMISSARIAT A L’ENERGIE ATOMIQUE ET AUX ENERGIES ALTERNATIVES, PARIS (FR)

(72) Inventor: Laurent DUSSOPT, Grenoble (FR)

(21) Appl. No.: 18/649,427

(22) Filed: Apr. 29, 2024

(30) Foreign Application Priority Data
Apr. 28, 2023 (FR) 2304354

(51) Int. Cl. G01N 21/45; G01N 33/02

(52) U.S. Cl. CPC G01N 21/45; G01N 33/02; G01N 2021/451

(57) ABSTRACT

An interferometric element intended for a device for detecting at least one compound having a resonant absorption over a predetermined spectral region centred on a resonance wavelength λᵣ, the interferometric element comprising a detection subset optimised for the resonance wavelength λᵣ and comprising: a plurality of optical cavities of Fabry-Perot type that are resonant at the resonance wavelength λᵣ, each cavity comprising a reflecting layer at the resonance wavelength λᵣ and a partially transparent layer at the resonance wavelength λᵣ, the partially transparent layer of the sensitive cavities being permeable to the compound or compounds to be detected an encapsulation layer that is impermeable to the compound or compounds to be detected and encapsulating a first subset of optical cavities, called reference cavities, such that each reference cavity has none of the compound to be detected between the reflecting layer and the partially transparent layer, the encapsulation layer not encapsulating a second subset of optical cavities, called sensitive cavities, such that each sensitive cavity can comprise the compound or compounds to be detected (C) between the reflecting layer and the partially transparent layer.

### CROSS-REFERENCE TO RELATED APPLICATION

This application claims priority to foreign French patent application No. FR 2304354, filed on Apr. 28, 2023, the disclosure of which is incorporated by reference in its entirety.

### FIELD OF THE INVENTION

The present invention relates to the field of the detection of particles and more particularly the detection of particles by optical interferometry.

### BACKGROUND

In many areas of application like the agri-food industry, defence or chemistry, the detection and the identification of particles are necessary in order to warn of a possible attack or contamination. Many techniques known to the person skilled in the art make it possible to determine a chemical composition of a sample.

For example, chemical sensors and biosensors make it possible to obtain a rapid detection and a real-time control of the interaction between the sample or the compounds to be detected and the sensor. Such sensors use a chemical or biomolecular detection layer in order to recognise a compound to be detected by bonding with the latter. This layer can for example comprise molecules like antibodies, enzymes, hormones, DNA, neurotransmitter receivers, etc.

These types of sensors cannot be reused because the step of bonding between the compound to be detected and the detection layer is generally not reversible. These sensors are therefore used once and then disposed of.

Integrated optical sensors provide an attractive alternative to these chemical sensors. Indeed, the techniques for manufacturing waveguides integrated on optical chips by photolithography and microfabrication allow for mass, inexpensive and repeatable production of integrated optical sensors. Most of these integrated optical sensors are interferometers of Mach-Zender type (or MZI for Mach-Zender interferometer).

Fourier transform infrared spectroscopy (FTIR) is an analytical technique that is extremely widely used in which the molecules of the sample absorb the incident radiation thus modifying their vibration energies. Based on the bonds and the chemical functions present in the sample, a characteristic infrared (IR) spectrum is obtained.

In addition to the FTIR spectroscopy, there are a multitude of techniques that make it possible to identify particles in a sample, such as Raman spectrometry, laser-induced plasma spectrometry (LIBS), or even the fluorescence-induced photo-fragmentation method (PF-LIF), specific to the NO2 bonds.

The techniques that involve infrared absorption can be differentiated according to several modalities.

A first method is the “passive” IR imaging in transmission or in backscattering, by direct absorption of the light by the particle. In this case, the optical power collected diminishes in the presence of the sample. It is for example known practice to analyse a gas generated by the breakdown of the sample subjected to an intense pulsed laser in mid-infrared (MIR) backscattering mode. The detected radiation is the thermal radiation (of black body type) of the objects that make up the observed scene. The latter is observed through an infrared imager, the detection spectral band of which is either wideband (covers all of the IR spectrum), or restricted to a part of the spectrum. An image processing uses the contrasts, both in the spectral and spatial domains, between the two types of images and deduces therefrom a level of absorption and therefore a gas concentration. This type of system is well suited to strong gas concentrations and vast scenes, for example for monitoring the emissions of an industrial site. This imaging technique makes it possible to detect and identify gas clouds. This technique cannot be used for objects of small dimensions like particles since the optical path travelled in the object is too small.

A second method consists in performing “active” thermal imaging. Indeed, there are active sensors that incorporate, in one module, an infrared source, filters and detectors that make it possible to detect a signal variation as a function of the presence of the gas sought. This type of system can access a very high level of sensitivity. On the other hand, it can be bulky, costly and requires a source of energy. These drawbacks can be prohibitive if it is sought to detect a gas in an environment that is confined, inaccessible, or that requires inexpensive technologies.

Finally, a last technique is photo-acoustics which can be separated into four steps:
(1) absorption of the laser radiation by the exciting gas as well as the rotational, electronic and vibrational energy levels;
(2) in the case of ro-vibrational excitations, de-excitation of the gas preferentially by molecular collisions which will be reflected by a transfer of rotation/vibration energy and kinetic energy creating a localised heating of the gas;
(3) generation of an acoustic wave and of a thermal wave provoked by the expansion due to the heating of the gas;
(4) detection by the microphone of the acoustic signal. The vibration amplitude of the microphone is representative of the concentration of the gas and the wavelength of the laser radiation absorbed by the gas indicates its composition.

This last method is interesting but it does not make it possible to have an image of a zone. It would be necessary to scan the laser over the sample which requires instrumentation and measurement time. Furthermore, this technique requires a laser source that can be modulated in intensity or in wavelength, which means that the detection device will be very expensive.

### SUMMARY OF THE INVENTION

The invention aims to mitigate certain problems of the prior art by providing a passive interferometric element that makes it possible to remotely detect a predetermined compound, possibly through a material wall that is transparent to the wavelength range used. The interferometric element of the invention is of particular interest for low-cost applications (for example the inspection of consumer products), or in industrial applications in hostile atmospheres (high or low temperatures, explosive or corrosive atmospheres).

To this end, one subject of the invention is an interferometric element intended for a device for detecting at least one compound having a resonant absorption over a predetermined spectral region centred on a resonance wavelength λᵣ, said interferometric element comprising at least one detection subset optimised for said resonance wavelength λᵣ and comprising:
at least two optical cavities of Fabry-Pérot type having a resonance at said resonance wavelength λᵣ, each cavity comprising a reflecting layer (CR) that is reflective to said resonance wavelength λᵣ and a layer that is partially transparent to said resonance wavelength λᵣ,
an encapsulation layer that is impermeable to the compound or compounds to be detected and encapsulating at least one optical cavity, called reference cavity, such that said reference cavity has none of said compound to be detected between the reflecting layer and the partially transparent layer, the encapsulation layer not encapsulating the second optical cavity, called sensitive layer, and the partially transparent layer of the sensitive cavities being permeable to the compound or compounds to be detected such that the sensitive cavity can comprise the compound or compounds to be detected between the reflecting layer and the partially transparent layer.

Preferentially, the interferometric element comprises a plurality of sensitive cavities and a plurality of reference cavities, and the sensitive cavities and the reference cavities are arranged according to a predetermined disposition so as to be able to determine a position of the sensitive cavities and of the reference cavities by the processing of an image of said interferometric element. More preferentially, said predetermined disposition is such that the sensitive cavities and the reference cavities are disposed alternately in a row or a plurality of rows, preferentially parallel.

According to one embodiment, the interferometric element comprises an optical pattern adapted so as to be able to determine an orientation and a position of said interferometric element by the processing of an image of said interferometric element.

According to one embodiment, the sensitive cavity or cavities are adapted to have a coefficient of reflection Rₛ(λᵣ) at the resonance wavelength λᵣ and the reference cavity or cavities are adapted to have a coefficient of reflection Rᵣ(λᵣ) at the resonance wavelength λᵣ such that Rᵣ(λᵣ)−Rₛ(λᵣ)>1%, and preferentially Rᵣ(λᵣ)−Rₛ(λᵣ)>2%, for a 1% concentration of the compound or compounds to be detected between the reflecting layer and the partially transparent layer of the sensitive cavities.

According to one embodiment, the interferometric element comprises a plurality of detection subsets each optimised for a respective resonance wavelength and different from the other resonance wavelength or wavelengths. Preferentially, the resonance wavelengths are less than 50% apart from one another. Alternatively, the resonance wavelengths are at least 5% apart from one another. According to a variant of this embodiment, the optical cavities of each detection subset comprise, between said partially transparent layer and said reflecting layer, an identical dielectric layer respectively associated with said detection subset, a refractive index and a thickness of said dielectric layer respectively associated with said detection subset being different from a refractive index and a thickness of the dielectric layer or layers respectively associated with the other detection subset or subsets and being adapted such that the optical cavities have a same thickness.

According to one embodiment, the partially transparent layer is separated by a distance p×λᵣ/2, from the reflecting layer, with p∈ [symbol] *>2, preferentially p>4.

Another subject of the invention is a device for detecting at least one compound having a resonant absorption over a spectral region centred on a resonance wavelength λᵣ, said device comprising:
a light source adapted to generate a first incident beam having at least said resonance wavelength λᵣ
an interferometric element according to the invention
arranged such that the first beam illuminates the partially transparent layer of the at least one sensitive cavity and the partially transparent layer of the at least one reference cavity
a sensor comprising a plurality of pixels and adapted to acquire an image of the first incident beam reflected by the interferometric element, called first image,
a processing unit linked to the sensor and configured to detect a possible presence of the compound or compounds to be detected from a comparison of an intensity between, on the one hand, at least one first region of pixels in which the first incident beam reflected by said at least one sensitive cavity is detected and, on the other hand, at least one second region of pixels in which the first incident beam reflected by said at least one reference cavity is detected.

Another subject of the invention is a device for detecting at least one compound having a resonant absorption over a spectral region centred on a resonance wavelength λᵣ, said device comprising
a light source adapted to generate a first incident beam having at least said resonance wavelength λᵣ and to generate a second incident beam not having any wavelength included in said resonant absorption and having at least one wavelength called non-resonance wavelength λₙᵣ,
an interferometric element according to the invention arranged such that the first beam and the second beam illuminate the partially transparent layer of at least one sensitive cavity and the transparent layer of at least one reference cavity
a sensor comprising a plurality of pixels and adapted to acquire an image of the first incident beam reflected by the interferometric element, called first image, and an image of the second incident beam reflected by the interferometric element, called second image
a processing unit linked to the sensor and configured to detect a possible presence of the compound or compounds to be detected from a comparison of an intensity of the first image and of the second image.

Another subject of the invention is a method for detecting at least one compound having a resonant absorption over a spectral region centred on a resonance wavelength λᵣ, said method comprising the following steps:
generating a first incident beam having at least said resonance wavelength λᵣ and a second incident beam not having any wavelength included in said resonant absorption and having at least one wavelength called non-resonance wavelength λₙᵣ,
illuminating an interferometric element according to the invention with the first beam and the second beam such that they illuminate the partially transparent layer of at least one sensitive cavity and the transparent layer of at least one reference cavity
acquiring an image of the first incident beam reflected by the interferometric element, called first image, and an image of the second incident beam reflected by the interferometric element, called second image
detecting a possible presence of the compound or compounds to be detected from a comparison of an intensity of the first image and of the second image.

According to one embodiment, said detection comprises the following steps:
A—calculating a third image by the difference between the second image and the first image
B—in the third image, comparing the intensity between, on the one hand, at least one first region of pixels in which the first incident beam reflected by said at least one sensitive cavity is detected, and, on the other hand, at least one second region of pixels in which the first incident beam reflected by said at least one reference cavity is detected.

Preferentially, the detection comprises a step A0, implemented before said step A, consisting in realigning the first image and the second image.

According to one embodiment, said detection is performed when an average Imoy₁ of the intensity of said at least one first region and an average Imoy₂ of the intensity of said at least one second region are such that:

$$\frac{\vert {Imoy}_{1} - {Imoy}_{2} \vert}{{Imoy}_{1}} > S ,$$

with S lying between 0.5% and 5%.

According to one embodiment, the first image and the second image are acquired simultaneously or within a time interval of less than 5 seconds, preferentially less than 1 second.

### BRIEF DESCRIPTION OF THE DRAWINGS

Other features, details and advantages of the invention will emerge on reading the description given with reference to the attached drawings that are given by way of example and which represent, respectively:

FIG. 1, a schematic view of a device 1 for detecting at least one compound according to the invention,

FIG. 2A, a cross-sectional view along a plane yz of an interferometric element according to an embodiment of the invention,

FIG. 2B, the trend of the coefficient of reflection of the sensitive cavities (curve C1) and of the reference cavities (curve C2) as a function of the wavelength,

FIG. 2C, the trend of the coefficient of reflection of the optical cavities as a function of the concentration of compound in the ambient medium of the interferometric element,

FIG. 3A, an example of the images acquired for an interferometric element according to an embodiment of the invention,

FIG. 3B, an interferometric element according to an embodiment of the invention,

FIG. 4, a graphic representation of the trend of the coefficient of reflection of a cavity according to an embodiment as a function of the wavelength, for five different H₂S concentration values in the cavity,

FIG. 5A, FIG. 5B, an example of a non-resonance image and an image with resonance respectively, and of the histograms of the pixels of these two images acquired for an interferometric element according to an embodiment of the invention,

FIG. 6, the trend of the value of the coefficient of reflection as a function of the loss tangent at resonance, for three different distances L,

FIG. 7, an embodiment in which the interferometric element comprises two detection subsets each optimised for a respective resonance wavelength and different from the other,

FIG. 8, an object of the invention which is a packaging, for example food packaging, comprising a food AL and comprising the interferometric element of the invention.

In the figures, unless stated otherwise, the elements are not to scale and identical references denote identical elements.

### DETAILED DESCRIPTION

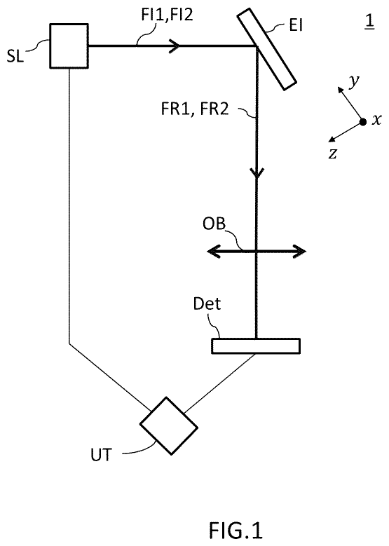

FIG. 1 illustrates in a simplified manner a device 1 according to the invention for detecting at least one compound C. The device 1 essentially comprises a light source SL, an interferometric element EI according to the invention, a sensor Det and a processing unit UT.

To simplify the description, it is assumed that the device 1 is optimised to detect a single compound C except when the reverse is explicitly specified. The compound to be detected C is known and predetermined and has a resonant absorption over a spectral region centred on a resonance wavelength λᵣ. As a nonlimiting example, the compound to be detected is hydrogen sulfide (H₂S). The absorption spectrum of the gas H₂S has several bands of high absorption in the terahertz domain, and in particular a line centred at 612 GHz. Works have shown the appearance of this gas and the increase in its concentration inside packages of food products in a few days, by making an indicator of the spoilage of the product (see L. Kuuliala et al., “Spoilage evaluation of raw Atlantic salmon (Salmo salar) stored under modified atmospheres by multivariate statistics and augmented ordinal regression”, International Journal of Food Microbiology, vol. 303, p. 46-57, August 2019, doi: 10.1016/j.ijfoodmicro.2019.04.011.).

The light source SL is adapted to generate at the very least a first incident beam FI1 having at least one resonance wavelength λᵣ. The light source SL is not specific to the invention and is for example a laser source.

According to a main embodiment illustrated in FIG. 1, the light source SL is furthermore adapted to generate a second incident beam FI2 having no wavelength lying within the resonant absorption of the compound C and having at least one wavelength called non-resonance wavelength λₙᵣ. The expression “a second incident beam FI2 having no wavelength lying within the resonant absorption of the compound C” is understood here to mean that the second beam has no wavelength lying within a spectral interval dependent on the spectral width of the resonant absorption, which is for example such as [0.98×λᵣ; 1.02×λᵣ], and preferentially no wavelength within a spectral interval such as [0.96×λᵣ;1.04×λᵣ].

According to a first alternative of the main embodiment, the light source SL is a source adapted to simultaneously generate the first beam and the second beam. For example, the light source SL comprises a monochromatic laser source emitting at the resonance wavelength λᵣ and comprises an optical architecture that makes it possible to generate at least the non-resonance wavelength λₙᵣ from the resonance wavelength λᵣ, typically by taking a portion of the first beam FI1 and by doubling it in frequency. Alternatively, the light source SL comprises two laser sources, respectively monochromatic at the non-resonance wavelength λₙᵣ and at the resonance wavelength Aᵣ. Alternatively, the source SL is a wideband source covering a spectral band that is sufficiently wide to encompass the non-resonance wavelength λₙᵣ and the resonance wavelength λᵣ.

According to a second alternative, the light source SL is able to generate the first beam and the second beam successively in time. For example, the source SL comprises a wavelength-tunable laser source or two laser sources operating alternately and respectively monochromatic at the non-resonance wavelength λₙᵣ and at the resonance wavelength λᵣ.

Alternatively, according to a secondary embodiment, the light source SL is adapted to generate only the first incident beam FI1. Hereinbelow, the operation of the device according to the main embodiment will be described first of all, followed by the operation of the device according to the secondary embodiment.

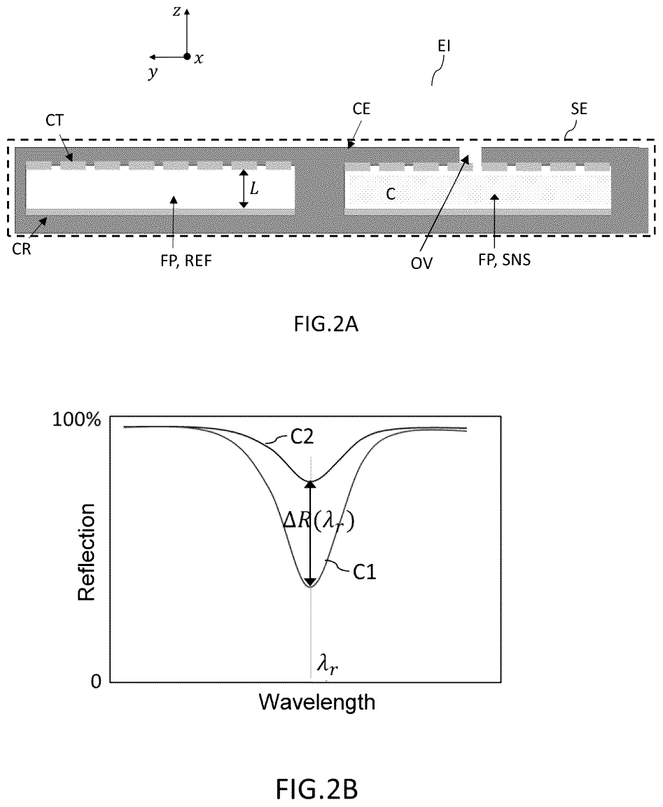

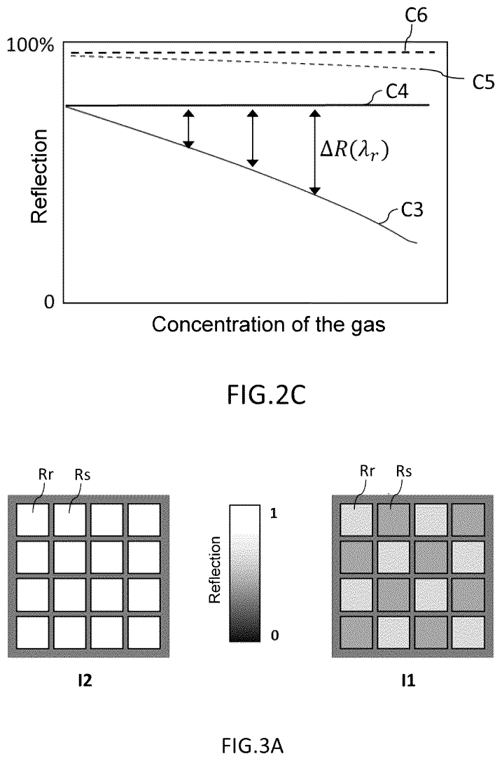

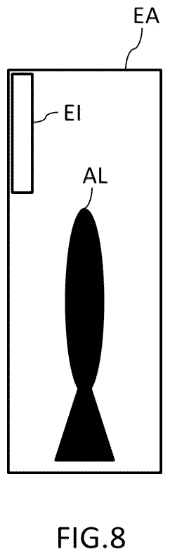

The interferometric element EI comprises at least one detection subset SE (not visible in FIG. 1, but visible in FIG. 2A) optimised for the resonance wavelength λᵣ so as to allow for the detection of the compound C. According to some embodiments, the interferometric element EI comprises several detection subsets each optimised to detect one or more respective compounds (see FIG. 7 described later). It is important to stress that the interferometric element EI of the invention is a passive element that requires no electrical power supply in order to allow the detection of the compound C.

FIG. 2A is a cross-sectional view along a plane yz of an interferometric element EI according to an embodiment of the invention.

The subset SE optimised to allow for the detection of the compound C comprises a plurality of optical cavities FP of Fabry-Pérot type having resonance at the resonance wavelength λᵣ. It is also necessary for the optical cavities FP not to have a resonant absorption at the non-resonance wavelength λₙᵣ. For that, for example, λₙᵣ will be chosen such that, for each resonance band of index i of the cavities FP, centred on a wavelength λ_{r,i} and of width at mid-height

$$δ {λ}_{i} , {λ}_{nr} ∉ [ {λ}_{r , i} - \frac{δ {λ}_{i}}{2} ; {λ}_{r , i} + \frac{δ {λ}_{i}}{2} ]$$

applies.

As a nonlimiting example, the subset SE of the interferometric element EI of FIG. 2A comprises two cavities FP aligned in the direction y.

Each of the cavities FP comprises a reflecting layer CR at the resonance wavelength λᵣ and a partially transparent layer CT at the resonance wavelength λᵣ. Typically, each reflecting layer CR has a coefficient of reflection greater than 99%, preferentially greater than 99.5%, at the resonance wavelength λᵣ and each partially transparent layer CT has a coefficient of reflection lying between 90% and 99%, preferentially lying between 97% and 99%, at the resonance wavelength λᵣ.

For example, the layers CT and CR are metallic layers, the layer CT being able to comprise a spatial structuring in order to increase its transmission.

Alternatively, the layer CT and/or the layer CR is or are a Bragg mirror composed of a stacking of dielectric materials of different optical indices and of thicknesses that are multiples of a quarter of the resonance wavelength.

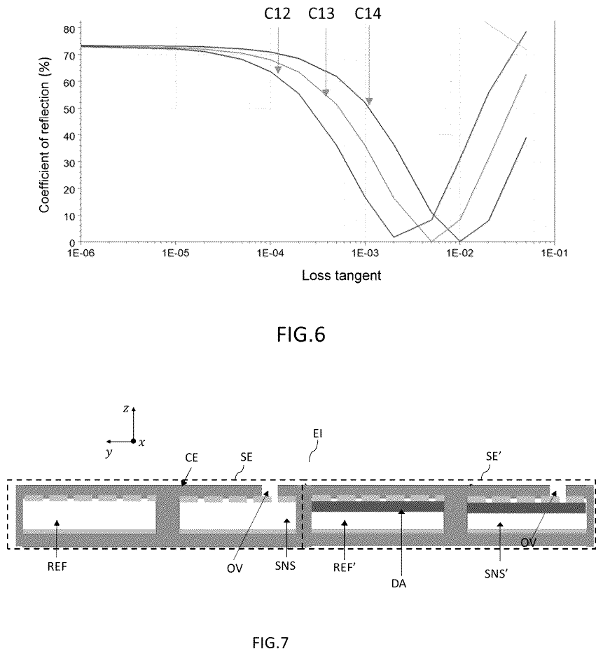

The layers CT and CR of each cavity are stacked in a direction of stacking (z in the illustration of FIG. 2A) so as to be separated by a distance L. This distance is adapted as a function of the medium or media separating the layers CT and CR for each cavity FP to have a resonance at the resonance wavelength λᵣ. As will be specified in more detail in FIG. 6, the greater this distance L, the greater the sensitivity of the sensor.

As is known, a Fabry-Perot interferometer has a path difference between each ray transmitted by the interferometer which has the value 2n·L·cos (θ), with e an angle of propagation of the rays between the layers CT and CR of the interferometer and with a medium of index n separating the layers CT and CR. It can be demonstrated that the transmission of a Fabry-Pérot interferometer is maximal when this path difference is equal to a multiple of the wavelength of the rays being propagated in the interferometer. In normal incidence, the transmission of each cavity FP is therefore maximal for a distance

$$L = \frac{p \times {λ}_{r}}{2 n} ,$$

with p∈ [symbol] *. Also, by assuming a medium of index n separating the layers CT and CR of the cavities FP of the interferometric element EI of the invention, the distance L of separation of the layers CT and CR is such that

$$L = \frac{p \times {λ}_{r}}{2 n} ,$$

with p∈ [symbol] *.

The subset SE of the interferometric element EI further comprises an encapsulation layer CE that is impermeable to the compound to be detected C and transparent to the wavelengths λᵣ and λₙᵣ. “Transparent” is understood here to mean that the encapsulation layer CE has a transmission greater than 80%, preferentially greater than 90%, for the wavelengths λᵣ and λₙᵣ.

This encapsulation layer CE is disposed so as to encapsulate a first subset of optical cavities, the reference cavity or cavities REF-, in such a way that each reference cavity has none of the compound to be detected between the layers CT and CR. Thus, according to one embodiment, the layers CT and CR of the reference cavity or cavities are separated by a vacuum or by a neutral medium (typically a gas that is non-resonant at the wavelengths λᵣ and λₙᵣ, for example nitrogen).

Furthermore, the encapsulation layer CE does not encapsulate a second subset of optical cavities: the sensitive cavity or cavities SNS. Thus, the sensitive cavity or cavities SNS of the interferometric element can comprise the compound to be detected C between their reflecting layer CR and their partially transparent layer CT when the ambient medium comprises the compound C. For example, as illustrated in FIG. 2A, the encapsulation layer CE comprises an opening OV so that the sensitive cavity SNS is exposed to the ambient medium. For that, it is necessary for the partially transparent layer CT of each sensitive cavity SNS to be permeable to the compound to be detected C.

Thus, the sensitive cavity or cavities and the reference cavity or cavities have a coefficient of reflection that is identical (or almost identical) and very high (typically greater than 99%) at the non-resonance wavelength λₙᵣ and have a coefficient of reflection that is lower and different at the resonance wavelength λᵣ. This result is illustrated in FIG. 2B which represents the trend of the coefficient of reflection of the sensitive cavities (curve C1) and of the reference cavities (curve C2) as a function of the wavelength.

On the curve C2, it can be seen that the reference cavities have a coefficient of reflection close to 100% over most of the spectrum and slightly less at the resonance wavelength λᵣ because of the higher losses generated by the phenomenon of electromagnetic resonance.

On the curve C1, it can be seen that the sensitive cavities also have a coefficient of reflection close to 100% over most of the spectrum because the compound C has no significant effect on the transmission in the absence of resonance. On the other hand, the coefficient of reflection is clearly reduced with respect to the reference cavity at the resonance wavelength λᵣ given the absorption of the compound C.

ΔR(λᵣ)=Rᵣ(λᵣ)−Rₛ(λᵣ) denotes the difference between the value Rₛ(λᵣ) of the coefficient of reflection at the resonance wavelength of the reference cavities λᵣ and the value Rᵣ(λᵣ) of the coefficient of reflection at the resonance wavelength of the sensitive cavities. It can be demonstrated that this difference Δ_{R}(λᵣ) is substantially proportional to the concentration of the compound C. This result can be observed in FIG. 2C which illustrates the trend of the coefficient of reflection of the optical cavities as a function of the concentration of compound C in the ambient medium of the interferometric element EI (and therefore between the layers CR and CT of the sensitive cavities).

The curves C3 and C5 of FIG. 2C respectively illustrate the trend of the coefficient of reflection of the sensitive cavities as a function of the concentration of compound C at the resonance wavelength and at the non-resonance wavelength.

The curves C4 and C6 of FIG. 2C illustrate the trend of the coefficient of reflection of the reference cavities as a function of the concentration of compound C at the resonance wavelength and at the non-resonance wavelength respectively.

The reference cavities have a coefficient of reflection that is constant or almost constant. The value of this coefficient of reflection is close to 100% and is substantially higher for the non-resonance wavelength.

The sensitive cavities have a coefficient of reflection that is high and slightly decreasing with the concentration of gas outside of resonance (C5) and a reflection that is strongly decreasing at resonance (C3).

Preferentially, the sensitive and reference cavities are adapted—through the coefficients of reflection of the layers CR and CT—to have a coefficient of reflection Rₛ(λᵣ) and coefficient of reflection Rᵣ(λᵣ) respectively such that ΔR(λᵣ)=Rᵣ(λᵣ)−Rₛ(λᵣ)>1%, and preferentially ΔR(λᵣ)>2%, for a 1% concentration of the compound to be detected between the reflecting layer and the partially transparent layer of the sensitive cavities. This value makes it possible to facilitate the detection of the compound C.

As illustrated in FIG. 1, the interferometric element EI is arranged so as to be illuminated by the first beam and the second beam FI1, FI2. More specifically, the beams FI1, FI2 illuminate the layer CT of at least one sensitive cavity SNS and the layer CT of at least one reference cavity REF.

The sensor Det comprises a plurality of pixels and is adapted to acquire an image of the first reflected incident beam FR1 reflected by the interferometric element, called first image I1 or resonance image I1. Furthermore, the sensor is able to acquire an image of the second reflected incident beam FR2 reflected by the interferometric element, called second image I2 or non-resonance image I2.

The sensor Det is not specific to the invention and will be adapted by the person skilled in the art according to the light source SL without departing from the scope of the invention.

For example, according to the embodiment in which the light source SL is a wideband source, the sensor Det comprises a matrix of pixels associated with a system of spectral filters capable of capturing images at λᵣ and at λₙᵣ. Typically, the system of spectral filters is a spectral filter wheel disposed optically upstream of the matrix of pixels. Alternatively, the sensor Det is a matrix of multispectral pixels and comprises, for example, several subsets of matrices of pixels each having a distinct spectral filter.

According to another embodiment in which the source emits a plurality of monochromatic beams successively or simultaneously, the sensor Det is for example a matrix of wideband pixels like a CCD or CMOS camera.

Finally, the processing unit UT is linked to the sensor Det and is configured to detect a possible presence of the compound to be detected C from a comparison of an intensity of the first image I1 and of the second image I2.

Thus, the passive interferometric element EI of the invention allows for the remote detection of the compound C. Depending on the resonance wavelength λᵣ used, this detection can be performed through an optically transparent element, for example a packaging of food type (see FIG. 8 described later). The interferometric element of the invention is particularly interesting for low-cost applications (for example the inspection of consumer products), or in industrial applications in a hostile atmosphere (high or low temperatures, explosive or corrosive atmospheres).

Indeed, as explained above, the structure of the interferometric element means that the regions of pixels where the reflection of the beams FR1, FR2 by the sensitive cavities are detected, called first regions Rs, have a different intensity between the first image and the second image I1, I2. Conversely, the regions of pixels where the reflection of the beams FR1, FR2 by the reference cavities are detected, called second regions Rr, have an equal or substantially equal intensity between the first image and the second image I1, I2. Through a comparison of the intensity of these regions Rs and Rr, the processing unit UT allows for a detection of the compound C in the sensitive cavities.

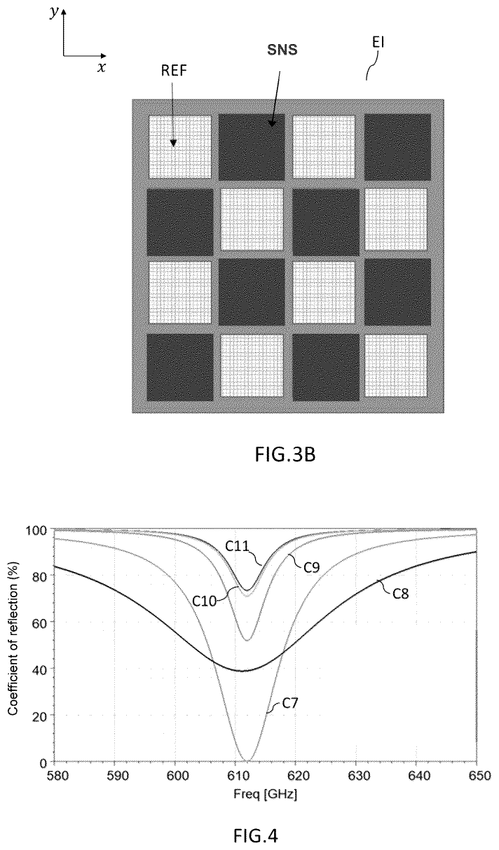

This result is illustrated in FIGS. 3A and 3B. More specifically, FIG. 3A is an example of the images I1 and I2 acquired for an interferometric element EI in the presence of the compound to be detected C in which the sensitive cavities SNS and reference cavities REF are arranged according to the illustration of FIG. 3B.

In the embodiment of FIG. 3B, as a nonlimiting example, the sensitive cavities SNS and reference cavities REF are disposed in “chequerboard” fashion, that is to say that the cavities are disposed alternately in a plurality of parallel rows. This disposition is advantageous because it allows for measurements of intensity in the images of the sensitive and reference cavities that are close and therefore associated with almost identical optical paths.

In FIG. 3A, it can be seen that the regions Rs and Rr associated respectively with the sensitive and reference cavities have an almost identical intensity in the non-resonance image I2 and have a different intensity in the resonance image I1. Indeed, given the presence of the compound C between the layers CT and CR of the sensitive cavities SNS which absorb the beam FI1 but not the beam FI2, the coefficient of reflection of the sensitive cavities SNS is less than that of the reference cavities REF for the beam FI1 (see FIG. 2B).

That is why, as illustrated in the image I1 of FIG. 3A, the intensity of the regions Rs is less than that of the regions Rr.

According to an embodiment of the main embodiment, the processing unit is adapted for the detection step to include a first step consisting in calculating a third image by the difference between the second image I2 and the first image I1.

Thus, provided that the images I1 and I2 are acquired simultaneously or within a sufficiently close time interval, the first step makes it possible to overcome the characteristics of the light source SL-interferometric element EI-sensor Det optical path (propagation losses, optical losses, orientation of the sensor, reflection/diffraction phenomena in the environment of the system, etc.). The calculation of the third image also makes it possible to locate the regions Rs and Rr more easily than in the images I1 and I2.

The expression “a sufficiently close time interval” is understood here to mean that the first image and the second image are acquired within a time interval of less than 5 seconds, preferentially less than 1 second.

In a second step, the processing unit UT is configured to compare the intensity between at least one first region Rs and at least one second region of pixels R2 in the third image. When the processing unit identifies a notable intensity difference between the region or regions Rs and the region or regions Rr, the processing unit UT considers the compound C to be detected.

Preferentially, the processing unit UT considers that the compound C is detected when an average Imoy₁ of the intensity of the first regions Rs and an average Imoy₂ of the intensity of the second regions Rr are such that:

$$Δ I = \frac{\vert {Imoy}_{1} - {Imoy}_{2} \vert}{{Imoy}_{1}} > S$$

with S a predetermined threshold dependent on:
the minimum concentration of compound C between the layers CT and CR that is wanted to be detected,
the coefficient of reflection of the sensitive cavities at λᵣ and λₙᵣ
the coefficient of reflection of the reference cavities at λᵣ and λₙᵣ.

As is known per se, the coefficients of reflection are determined by the coefficients of the layers CT and CR.

Typically, this threshold S lies between 0.5% and 5% for a maximum concentration of 1%.

Preferentially, the first step of calculation of the third image is preferentially preceded by a preliminary step of realignment between the first image and the second image. This realignment step is known per se and can be implemented by any method known to the person skilled in the art. In image processing, realignment is a technique which consists in “matching images”, in order to compare them or combine them.

According to one embodiment, this realignment step is performed by image processing methods based on the 2D correlation of the images I1 and I2.

According to one embodiment, compatible with all the embodiments of the invention, the sensitive cavities and the reference cavities are arranged according to a predetermined disposition so as to be able to determine a position of the sensitive cavities and of the reference cavities by image processing. Thus, the realignment step is implemented by the processing unit by image processing, by virtue of this predetermined disposition previously stored in the processing unit UT.

According to another embodiment, compatible with all the embodiments of the invention, the interferometric element comprises an optical pattern. This optical pattern is adapted so as to be able to determine an orientation and a position of the interferometric element from an image. The optical pattern thus facilitates the realignment of the images I1 and I2.

Preferentially, the interferometric element EI comprises a plurality of sensitive cavities and of reference cavities (for example more than five sensitive cavities and more than five reference cavities). That makes it possible to take an average of the regions Rs and Rr in the images I1 and I2 (or directly in the third image) and thus perform the step of comparison of intensity of the regions Rs and Rr implemented by the processing unit UT on the average of the regions Rs and the average of the regions Rr. Thus, it is possible to overcome the dispersions originating from the detectors, from the resonant cavities or from the environment (for example an object blanking out a part of the interferometric element).

According to one embodiment, the processing unit UT is further configured to determine the concentration of compound C, from the value of ΔI. Indeed, as explained previously, the difference λR(λᵣ) between the value of the coefficient of reflection between the sensitive cavities SNS and that of the reference cavities REF, at the resonance wavelength, is substantially proportional to the concentration of the compound C. Naturally, this difference λR(λᵣ) is proportional to the value of ΔI. It is therefore possible to perform a calibration by preliminary measurements with predetermined concentrations of compound C and thus associate a value of ΔI with a concentration of compound C.

As explained previously, in the secondary embodiment, the source SL is adapted to emit only the beam FI1 having the resonance wavelength λᵣ. Also, in the secondary embodiment, the sensor Det performs the acquisition of a single image: the image I1. The processing unit UT is then configured to detect a possible presence of the compound to be detected C directly from the first image I1, by a comparison of an intensity between at least one region Rs and at least one region Rr. Alternatively, as in the main embodiment, in the secondary embodiment this detection can be implemented by a comparison of an intensity between an average of the regions Rs and an average of the regions Rr.

By comparison to the main embodiment, the secondary embodiment offers the advantage of being easier to implement because the interferometric element EI is interrogated by using a single beam FI1. It is therefore possible to use a monochromatic laser source for example. Indeed, in theory, it is possible to perform the detection of the compound C by analysing only the image I1 in very good observation conditions. That presupposes that the difference of reflectivity between sensitive cavities and the reference cavities at resonance is sufficient to locate the regions Rs and Rr in the image I1 and that their position in the image I1 is known. However, in practice, the signal-to-noise ratio in the image I1 will be low given the uncontrolled and/or random spatial variations of the scene imaged by the detector. Thus, the detection of the compound C is more difficult in the secondary embodiment and the rate of false negative detections of the compound C will be higher than in the main embodiment.

Furthermore, in the main embodiment, the image I2 is advantageous because it greatly facilitates the identification of the regions Rs and Rr in the image I1.

### Example of Implementation of the Interferometric Element:

According to one embodiment of the invention, denoted MR1, the interferometric element EI comprises cavities FP that are optimised in order to have a resonance at the resonance wavelength λᵣ=612 GHz which is the central wavelength of a line of absorption of the gas H₂S.

In this embodiment, the reflecting layers CR are produced by a metallic layer. The partially transparent layers CT each comprise a stack composed of a bottom layer of silicon 5 μm thick, an intermediate layer comprising a matrix of 47×47 μm metal blocks with a pitch of 50 μm, and a top layer of silicon oxide 2 μm thick. The layers CT and CR are separated by a distance L=250 μm. This distance value L is close to λᵣ/2 (245 μm) but not strictly equal because of the phase of transmission of the layers of the stacking of the layer CT.

Preferably, the metallic elements are produced in a metal that is a good conductor, such as copper, gold or, by default, aluminium, in order to minimise the associated losses.

FIG. 4 is a graphic representation of the trend of the coefficient of reflection of a cavity according to the embodiment MR1 as a function of the wavelength, for five different values of concentration of H₂S in the cavity. More specifically, the layer C7 is obtained by a tangent of dielectric losses (tan δ) of 0.01, the curve C8 is obtained by a tangent of dielectric losses (tan δ) of 0.05, the curve C9 is obtained by a tangent of dielectric losses (tan δ) of 0.001, the curve C10 is obtained by a tangent of dielectric losses (tan δ) of 0.0001 and the curve C11 is obtained by a tangent of dielectric losses (tan δ) that is zero.

To recap, the tangent of dielectric losses is directly linked to the absorbance a of the compound C by the following relationship:

$$α = \frac{π}{λ} \sqrt{{ε}^{′}} tan δ ,$$

In which ε′ is the permittivity of the compound C. Thus, the loss tangent is proportional to the concentration of species C.

In FIG. 4, it can be seen that the coefficient of reflection shows a minimum at λᵣ=612 GHz, of a depth increasing with the tangent of losses, or increasing with the concentration of gas. The non-resonance coefficient of reflection, below 590 GHz or above 630 GHz, is close to 100% for low or moderate gas concentrations. On the curve C7, it can be seen that the coefficient of reflection at resonance is zero. It will therefore be potentially easy to identify the regions Rs in the image I1 for this concentration.

Through simulations calculating the trend of the non-resonance and resonance coefficients of reflection as a function of the concentration of gas, the values of reflection at resonance and non-resonance are determined for a concentration of gas that is zero and 1% presented in the following table (hereinafter “table 1”):

| tanδ | 0 | 7.2e−5 |
|---|---|---|
| Concentration (%) | 0 | 1% |
| Refl. 580 GHz (%) | 99.35% | 99.32% |
| Refl. at resonance (%) | 73.29% | 71.51% |

From these reflection values, it is possible to simulate the images obtained by a matrix of detectors having a noise level of 250 μVrms (typical value) and a signal-to-noise ratio (SNR) of 10. As a nonlimiting example, the element EI of the embodiment MR1 comprises a matrix of 5×5 cavities FR with, among them, 13 reference cavities and 12 sensitive cavities arranged in chequerboard fashion.

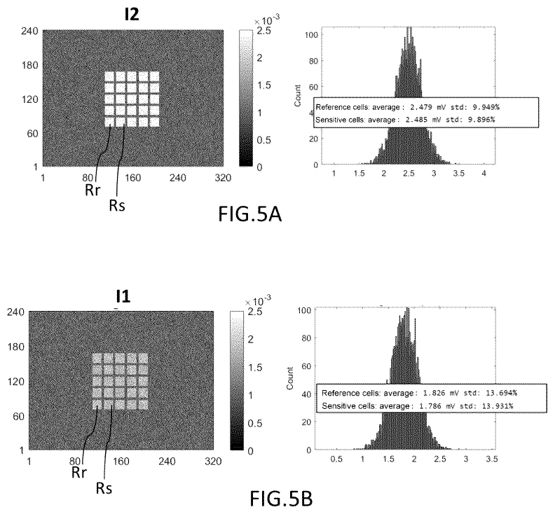

FIGS. 5A and 5B present the results of this simulation. More specifically, FIG. 5A presents the image I2 on the left and presents the histogram of the values of the pixels of the regions Rs and Rr in the image I2. FIG. 5B presents the image I1 on the left and presents the histogram of the values of the pixels of the regions Rs and Rr in the image I1. As an example, the regions Rs and Rr are each composed of approximately 3000 pixels.

It can be seen first of all that, in the images I1 and I2, it is not possible to distinguish the two types of cavities by the naked eye and therefore detect the regions Rs and Rr. On the other hand, it is possible to plot their histogram and calculate their average values.

In the histogram of FIG. 5A, it can be seen that the average value of the pixels of the region Rs has the value 2.485 mV and is substantially equal to the average value of the pixels of the region Rr which has the value 2.479 mV. Indeed, at non-resonance, the coefficient of reflections of the sensitive cavities and that of the reference cavities are almost equal (see table 1).

In the histogram of FIG. 5B, it can be seen that the average value of the pixels of the region Rs has the value 1.786 mV and is substantially less than the average value of the pixels of the region Rr which has the value 1.826 mV. Indeed, at resonance, the coefficient of reflections of the sensitive cavities is less than that of the reference cavities given the absorption of the compound C having a concentration of 1% (see table 1).

Here, the detection of the compound C can therefore be performed from the image I1 only by comparison of the average intensity of the regions Rs and Rr or else by calculating the third image from the difference between the image I2 and 11, then by comparing the average intensity of the regions Rs and Rr in this third image.

FIG. 6 illustrates the trend of the value of the coefficient of reflection as a function of the tangent of losses at resonance, for three different distances L in an optical cavity according to the embodiment MR1. More specifically, the curve C12 is obtained for L=2·λᵣ, the curve C13 is obtained for L=λᵣ and the curve C14 is obtained for L=λᵣ/2.

FIG. 6 makes it possible to observe that the choice of a distance L that is relatively greater makes it possible to improve the sensitivity of the interferometric element EI because a significant variation of reflection will be obtained for a lower loss tangent (i.e. concentration of gas). That is explained by the fact that a greater distance L makes it possible to have a greater quantity of gas in the cavity.

Also, preferentially, the distance L of separation between the partially transparent layer and the reflecting layer in the cavities FP of the interferometer of the invention is such that L=p×λᵣ/2 with p∈ [symbol] *>2, preferentially p>4. That makes it possible to improve the sensitivity of the interferometric element EI.

FIG. 7 illustrates an embodiment in which the interferometric element EI comprises two detection subsets SE and SE′, each optimised for a respective resonance wavelength and different from the other. Thus, the interferometric element EI of FIG. 7 makes it possible to detect two different compounds C or allows for a detection of two different resonances of a same gas.

More generally, according to one embodiment, the interferometric element EI comprises a plurality of detection subsets, each optimised for a respective resonance wavelength and different from the other resonance wavelength or wavelengths.

Preferentially, the resonance wavelengths are less than 50% apart from one another. Thus, the manufacturing uncertainties can be overcome.

Alternatively, the resonance wavelengths are at least 5% apart from one another. Thus, it is possible to detect several different compounds C or several different resonances of a same compound.

As illustrated in FIG. 7, the optical cavities FP of a subset SE can comprise an identical dielectric layer DA disposed between the layers CT and CR. This dielectric layer DA is preferentially formed from a material with low losses in order to reduce the thickness between the two surfaces by increasing the mean refractive index of the medium situated between the layers CT and CR.

Preferably, the refractive index and the thickness of the dielectric layer DA are adapted in such a way that the optical cavities have a same thickness (that is to say a same dimension in the direction of stacking). That makes it possible to facilitate the manufacturing of the interferometric element.

FIG. 8 illustrates an object of the invention which is a packaging EA, for example food packaging including a food AL. This packaging EA comprises the interferometric element EI of the invention in the packaging in the volume where the food AL is conserved and disposed. As a nonlimiting example, the structure of each of the cavities FP of the interferometric element EI is optimised to have a resonance with a resonant absorption of the gas H₂S, such as 612 GHz because the detection of hydrogen sulfide in the perishable food products such as meats or fresh fish is an indicator of the spoilage of the product. Thus, when the interferometric element EI is interrogated by a light source SL emitting at least at the wavelength λᵣ=612 GHz, it will be possible to detect a possible alteration of the food AL by a presence of the gas H₂S.

It is preferable for the light source SL to be adapted as a function of the food packaging in order for the latter to have a sufficient transmission (for example greater than 50%) at the wavelength λᵣ. For example, in the terahertz range, it will be possible to pass through a wall made of plastic, paper, cardboard, or even fabric.

1. An interferometric element (EI) intended for a device for detecting at least one compound (C) having a resonant absorption over a predetermined spectral region centred on a resonance wavelength λᵣ, said interferometric element comprising at least one detection subset (SE) that is optimised for said resonance wavelength λᵣ and comprising:
at least two optical cavities of Fabry-Pérot (FP) type having a resonance at said resonance wavelength λᵣ, each cavity comprising a reflecting layer (CR) at said resonance wavelength λᵣ and a partially transparent layer (CT) at said resonance wavelength λᵣ,
an encapsulation layer (CE) that is impermeable to the compound or compounds to be detected (C) and encapsulating at least one optical cavity, called reference cavity (REF), such that said reference cavity has none of said compound to be detected between the reflecting layer (CR) and the partially transparent layer, the encapsulation layer not encapsulating the second optical cavity, called sensitive cavity (SNS), and the partially transparent layer of the sensitive cavities being permeable to the compound or compounds to be detected (C) such that the sensitive cavity can include the compound or compounds to be detected (C) between the reflecting layer (CR) and the partially transparent layer.

2. The interferometric element according to claim 1, comprising a plurality of sensitive cavities and a plurality of reference cavities, wherein the sensitive cavities and the reference cavities are arranged according to a predetermined disposition so as to be able to determine a position of the sensitive cavities and of the reference cavities by the processing of an image of said interferometric element.

3. The interferometric element according to claim 2, wherein said predetermined disposition is such that the sensitive cavities and the reference cavities are disposed alternately along a row or a plurality of rows, preferentially parallel.

4. The interferometric element according to claim 1, comprising an optical pattern adapted so as to be able to determine an orientation and a position of said interferometric element by the processing of an image of said interferometric element.

5. The interferometric element according to claim 1, wherein the sensitive cavity or cavities are adapted to have a coefficient of reflection Rₛ(λᵣ) at the resonance wavelength λᵣ and the reference cavity or cavities are adapted to have a coefficient of reflection Rᵣ(λᵣ) at the resonance wavelength λᵣ such that Rᵣ(λᵣ)−Rₛ(λᵣ)>1%, and preferentially Rᵣ(λᵣ)−Rₛ(λᵣ)>2%, for a 1% concentration of the compound or compounds to be detected (C) between the reflecting layer (CR) and the partially transparent layer of the sensitive cavities.

6. The interferometric element according to claim 1, comprising a plurality of detection subsets (SE, SE′) each optimised for a respective resonance wavelength and different from the other resonance wavelength or wavelengths.

7. The interferometric element according to claim 6, wherein the resonance wavelengths are less than 50% apart from one another.

8. The interferometric element according to claim 6, wherein the resonance wavelengths are at least 5% apart from one another.

9. The interferometric element according to claim 6, wherein the optical cavities of each detection subset comprise, between said partially transparent layer and said reflecting layer, an identical dielectric layer (DA) respectively associated with said detection subset, a refractive index and a thickness of said dielectric layer respectively associated with said detection subset being different from a refractive index and a thickness of the dielectric layer or layers respectively associated with the other detection subset or subsets and being adapted in such a way that the optical cavities have a same thickness.

10. The interferometric element according to claim 1, wherein the partially transparent layer is separated by a distance p×λᵣ/2, from the reflecting layer, with p∈ [symbol] *>2, preferentially p>4.

11. A use of said interferometric element according to claim 1, disposed in a packaging comprising a food, for detecting an alteration of said food.

12. A device for detecting at least one compound (C) having a resonant absorption over a spectral region centred on a resonance wavelength λᵣ, said device comprising:
a light source (SL) adapted to generate a first incident beam (FI1) having at least said resonance wavelength λᵣ
an interferometric element (EI) according to claim 1, arranged such that the first beam illuminates the partially transparent layer and the at least one sensitive cavity and the partially transparent layer of the at least one reference cavity
a sensor (Det) comprising a plurality of pixels and adapted to acquire an image of the first incident beam reflected by the interferometric element, called first image,
a processing unit (UT) linked to the sensor and configured to detect a possible presence of the compound or compounds to be detected (C) from a comparison of an intensity between, on the one hand, at least one first region of pixels where the first incident beam reflected by said at least one sensitive cavity is detected and, on the other hand, at least one second region of pixels where the first incident beam reflected by said at least one reference cavity is detected.

13. The device for detecting at least one compound (C) having a resonant absorption over a spectral region centred on a resonance wavelength λᵣ, said device comprising
a light source (SL) adapted to generate a first incident beam (FI1) having at least said resonance wavelength λᵣ and to generate a second incident beam (FI2) having no wavelength included in said resonant absorption and having at least one wavelength called non-resonance wavelength λₙᵣ, an interferometric element (EI) according to claim 1, arranged such that the first beam and the second beam illuminate the partially transparent layer of at least one sensitive cavity and the transparent layer of at least one reference cavity
a sensor (Det) comprising a plurality of pixels and adapted to acquire an image of the first incident beam reflected by the interferometric element (FR1), called first image (I1), and an image of the second incident beam reflected by the interferometric element (FR2), called second image (I2) a processing unit (UT) linked to the sensor and configured to detect a possible presence of the compound or compounds to be detected (C) from a comparison of an intensity of the first image and of the second image.

14. A method for detecting at least one compound (C) having a resonant absorption over a spectral region centred on a resonance wavelength λᵣ, said method comprising the following steps:
generating a first incident beam (FI1) having at least said resonance wavelength λᵣ and a second incident beam (FI2) having no wavelength included in said resonant absorption and having at least one wavelength called non-resonance wavelength λₙᵣ,
illuminating an interferometric element (EI) according to claim 1 with the first beam and the second beam such that they illuminate the partially transparent layer of at least one sensitive cavity and the transparent layer of at least one reference cavity
acquiring an image of the first incident beam reflected by the interferometric element, called first image, and an image of the second incident beam reflected by the interferometric element, called second image
detecting a possible presence of the compound or compounds to be detected (C) from a comparison of an intensity of the first image and of the second image.

15. The method according to claim 14, wherein said detection comprises the following steps:
A. calculating a third image by the difference between the second image and the first image
B. in the third image, comparing the intensity between, on the one hand, at least one first region of pixels where the first incident beam reflected by said at least one sensitive cavity is detected, and, on the other hand, at least one second region of pixels where the first incident beam reflected by said at least one reference cavity is detected.

16. The method according to claim 15, wherein said detection comprises a step A0, implemented before said step A, consisting in realigning the first image and the second image.

17. The method according to claim 15, wherein said detection is performed when an average Imoy₁ of the intensity of said at least one first region and an average Imoy₂ of the intensity of said at least one second region are such that:
$$\frac{\vert {Imoy}_{1} - {Imoy}_{2} \vert}{{Imoy}_{1}} > S ,$$
with S lying between 0.5% and 5%.

18. The method according to claim 15, wherein the first image and the second image are acquired simultaneously or within a time interval of less than 5 seconds, preferentially less than 1 second.
